(19) United States
(12) Patent Application Publication
(10) Pub. No.: US 2022/0117073 A1
(43) Pub. Date: Apr. 14, 2022

(54) PLASMA TREATMENT DEVICE WITH BRUSH HEAD

(71) Applicant: CINOGY GMBH, Duderstadt (DE)

(72) Inventors: Dirk WANDKE, Heiligenstadt (DE); Mirko HAHNL, Berlingerode (DE); Karl-Otto STORCK, Duderstadt (DE); Leonhard TRUTWIG, Duderstadt/Gerblingerode (DE); Melanie RICKE, Katlenburg-Lindau (DE)

(21) Appl. No.: 17/287,605

(22) PCT Filed: Oct. 23, 2019
(86) PCT No.: PCT/EP2019/078893

§ 371 Date: Aug. 23, 2021

(30) Foreign Application Priority Data
Oct. 24, 2018 (DE) 10 2018 126 489.5

(51) Int. Cl. H05H 1/24; A61N 1/44

(52) U.S. Cl. CPC H05H 1/2406; A61N 1/44; A46B 2200/10; H05H 2277/10; H05H 2245/40

(57) ABSTRACT

A plasma treatment device (1), designed for treating a surface with a dielectrically impeded plasma, comprising an electrode arrangement (5) which has at least one electrode (3), and comprising dielectric (7) which completely covers the electrode (3) in relation to the surface to be treated, and comprising a housing (9) which contains a line arrangement (11) which comprises at least one high-voltage supply line (10, 10a, 10b), wherein the electrode (3) is connected to the line arrangement (11) and can be acted on by a high-voltage signal (13a, 13b), which can be applied to the high-voltage supply line (10, 10a, 10b), via the high-voltage supply line (10, 10a, 10b), allows, in a simple manner, the combination of an effective plasma treatment with an effective mechanical treatment of the surface to be treated by way of the plasma treatment device (1) having a brush head (15) which has a bristle area (17) and a bristle support (19) with a base surface (21), wherein the bristle area (17) has a large number of flexible bristles (23) and interspaces between the bristles (23), and the bristles (23) protrude from the base surface (21) of the bristle support (19) in a direction of an abutment surface (27) which is defined by those ends of the longest bristles (23) of the bristle area (17) that are averted from the base surface (21), wherein the bristle area (17) has a first length which is defined by the distance between the base surface (21) and the abutment surface (27), and wherein the at least one electrode (3) of the electrode arrangement (5), starting from the base surface (21), extends with a second length, which is smaller than the first length or equal to the first length and is at least 30% of the first length, into the bristle area (17) in the direction of the abutment surface (27).

The invention relates to a plasma treatment device designed to treat a surface with a dielectric barrier plasma, having an electrode arrangement that has at least one electrode and having a dielectric that completely covers the electrode in the direction of the surface to be treated, and having a housing that contains a line arrangement comprising at least one high-voltage supply line, wherein the electrode is connected to the line arrangement and is able to be supplied, via the high-voltage supply line, with a high-voltage signal able to be applied to the high-voltage supply line.

The at least one high-voltage supply line is able to be connected or is connected to a high voltage required for plasma generation, which high voltage is preferably used as an AC high voltage. The electrode may thereby be supplied with a high-voltage signal via the high-voltage supply line, which high-voltage signal is preferably in the form of an AC high-voltage signal.

The plasma treatment device according to the invention may be designed to be connected to an external high-voltage source. However, it is preferable for the plasma treatment device to be designed to generate the high voltage in the plasma treatment device itself. A normal supply voltage (for example 230 V or 110 V mains voltage) may in this case be supplied to the plasma treatment device. For this purpose, the housing of the plasma treatment device may contain a high-voltage stage for generating a high voltage, for example a high-voltage generator, which is connected at output to the at least one high-voltage supply line of the line arrangement. Such a structure of the plasma treatment device according to the invention offers the advantage that no high voltage has to be carried outside the device, such that it is significantly easier to guarantee the required safety of the device.

The housing of the plasma treatment device according to the invention may have a through-opening for the passage of a connection cable. The plasma treatment device may be supplied with a high voltage or, preferably, with a normal supply voltage of the abovementioned type via this connection cable.

In one advantageous embodiment of the invention, the plasma treatment device may however also have a battery that provides the required supply voltage. The battery may in this case be a rechargeable battery (accumulator) or a non-rechargeable battery. The housing of the plasma treatment device according to the invention may advantageously in particular contain the battery. The high-voltage stage is able to generate the required high voltage from the supply voltage provided by the battery.

The electrode arrangement of the plasma treatment device may advantageously in particular be configured such that the surface to be treated is used as counter-electrode. To this end, the surface to be treated has to be the surface of an electrically conductive body. Such an electrically conductive body may be for example a human or animal body whose skin surface is to be treated, or another electrically conductive body.

The surface to be treated or the associated body may in this case function as what is known as a floating counter-electrode. Such a floating counter-electrode follows the change in potential of the high-voltage signal applied to the electrode only very slowly. If an AC high-voltage signal is used as high-voltage signal, then the potential of the floating counter-electrode remains substantially at an average potential, which will generally be the reference potential. The surface to be treated or the associated body may therefore function as ground.

By way of example, it is possible to use only a single electrode to generate the plasma and to use the surface or the associated body as counter-electrode (ground). This advantageously achieves a large treatment depth within the body.

It is also possible for example to provide a plurality of electrodes, that is to say at least two electrodes, in the electrode arrangement, which are supplied with the same high-voltage signal. The surface to be treated in this case functions as counter-electrode for the plasma formation. As an alternative, it is possible for example to supply the at least two electrodes of the electrode arrangement with different AC high-voltage signals, in particular with AC high-voltage signals in phase opposition to one another. In this case too, the surface to be treated may again function as counter-electrode.

However, it is also possible for example to use the at least two electrodes of the plurality of electrodes as electrode and counter-electrode, such that the plasma arises between the electrodes and is able to act as surface plasma. Only small treatment depths are thereby possible with a normal energy input, however.

For a plasma treatment device of the type mentioned at the outset, it is essential for the dielectric to completely cover the at least one electrode in the direction of the surface to be treated. The at least one electrode is thereby shielded from the surface to be treated. Due to the fact that the dielectric completely covers the at least one electrode in the direction of the surface to be treated, a direct or galvanic current flow between the at least one electrode and the surface to be treated is prevented.

The dielectric or parts of the dielectric may in particular be designed to be flexible. Suitable materials for producing the flexible dielectric are for example flexible silicones, in particular silicone rubbers.

DE 10 2015 111 401 B3 discloses a treatment device for treating a surface with a dielectric barrier plasma. The treatment device has a housing, having an end wall, and an electrode. The electrode is able to be connected to a high-voltage generator and is shielded in the direction of the surface to be treated by a dielectric forming at least part of the end wall. The end wall has at least one spacer that forms at least one gas compartment when the spacer bears on the surface to be treated, in which gas compartment the dielectric barrier plasma for the treatment is formed.

DE 10 2012 015 482 A1 furthermore discloses an electrode arrangement for forming a dielectric barrier plasma between an active surface of the electrode arrangement and a surface functioning as counter-electrode. This electrode arrangement has a flexible, flat electrode that is able to be connected to a high-voltage source, and a flat, flexible dielectric that forms the active surface and that is connected to the flat electrode so as to form an electrode element and completely covers the electrode in the direction of the surface to be treated. In order to improve adaptability to uneven surfaces, this electrode arrangement furthermore has a surface-elastic pressure means on the rear side of the electrode element that faces away from the surface, by way of which the flat electrode element is pressed evenly in the direction of the surface to be treated.

The possible uses of such devices for treating surfaces with a dielectric barrier plasma are extremely varied. They lie in particular in the therapeutic and cosmetic sector, but are by no means restricted thereto. The known devices for treating surfaces with a dielectric barrier plasma have proven themselves and are suitable in particular for treating the skin surface of a human or animal body. The plasma treatment may for example improve the take-up of therapeutic or cosmetic active ingredients, such that the plasma treatment enhances the desired therapeutic or cosmetic effect. The plasma treatment furthermore ensures effective germ reduction, since it destroys microorganisms and in particular exerts a bactericidal and fungicidal effect on the skin. The plasma treatment also leads to an increase in microcirculation in tissue.

In many of the diverse applications of such plasma treatments, it is desirable to be able to combine the plasma treatment with a mechanical treatment of the surface, for example in order to achieve a cleaning and/or massage effect. The abovementioned previously known plasma treatment devices however do not allow a mechanical treatment of a surface of the abovementioned type in order to achieve a cleaning and/or massage effect.

Taking this as a starting point, the present invention is based on the object of improving the proven, previously known plasma treatment devices such that they allow both an effective plasma treatment and an effective mechanical treatment of the surface to be treated in order to achieve a cleaning and/or massage effect.

In order to achieve this object, a plasma treatment device of the type mentioned at the outset is characterized, according to the invention, in that the plasma treatment device has a brush head that has a bristle field and a bristle carrier having a base surface. The bristle field in this case has a multiplicity of flexible bristles and intermediate spaces between the bristles, and the bristles protrude from the base surface of the bristle carrier in the direction of a contact surface that is defined by the ends of the longest bristles of the bristle field that face away from the base surface. The bristle field has a first length defined by the distance between the base surface and the contact surface, and the at least one electrode of the electrode arrangement extends, starting from the base surface, with a second length that is less than the first length or equal to the first length and at least 30% of the first length, into the bristle field in the direction of the contact surface.

The plasma treatment device according to the invention, by virtue of its brush head and the flexible bristles of the bristle field, advantageously allows a plasma treatment to be combined with a mechanical treatment of the surface to be treated. The plasma treatment may thereby be integrated into an operation for example involving cleaning and/or massaging of the surface to be treated.

The plasma treatment and the mechanical treatment of the surface to be treated with the plasma treatment device according to the invention may in this case be performed simultaneously. The plasma treatment and the mechanical treatment may however in principle also take place in succession in any chronological order. The plasma treatment device may accordingly be designed to perform a plasma treatment and a mechanical treatment of the surface to be treated simultaneously and/or in succession.

One exemplary field of application of the plasma treatment device according to the invention is in the skin care sector. By way of example, it is advantageously possible, in connection with a plasma treatment of a skin surface of a human or animal body, to clean and/or to massage the skin surface. The mechanical treatment of a skin surface with the plasma treatment device according to the invention may in particular advantageously be used to stimulate blood flow. The plasma treatment device according to the invention may advantageously also be used for example to combine a plasma treatment with peeling of the skin.

The mechanical treatment of the surface may also improve the effectiveness of the plasma treatment, for example by virtue of opening the pores of the surface to be treated through the mechanical action. When treating a skin surface, stimulating blood flow may contribute to improving the effectiveness of the plasma treatment.

The treatment of the surface to be treated with the plasma treatment device according to the invention may advantageously be performed using therapeutic and/or cosmetic active ingredients. The treatment of the surface to be treated with the plasma treatment device according to the invention may advantageously be performed for example using a cleansing cream and/or a care cream. The treatment of the surface to be treated may however advantageously also be performed dry and/or without using therapeutic or cosmetic active ingredients, cleansing creams or care creams.

The ends of the longest bristles of the bristle field that face away from the base surface define a contact surface that bears on the surface to be treated when the plasma treatment device is used as intended. The bristles of the bristle field may in this case for example have a uniform length. In this case, the contact surface is defined by the ends of all of the bristles of the bristle field that face away from the base surface. As an alternative, the bristles of the bristle field may however also have different lengths, that is to say some bristles of the bristle field may be shorter than other bristles of the bristle field. In this case, the contact surface is defined by the ends, facing away from the base surface, of those bristles of the bristle field that have the greatest length and thus always bear on the surface to be treated when the plasma treatment device is used as intended.

In addition to effective mechanical treatment, the plasma treatment device according to the invention advantageously achieves an effective plasma treatment of the surface to be treated due to the fact that the at least one electrode of the electrode arrangement extends into the bristle field. During intended use of the plasma treatment device, that is to say during the treatment of the surface, a small distance between the electrode extending into the bristle field and the surface to be treated may thereby be guaranteed, regardless of the length of the bristles. By virtue of the small distance between the at least one electrode and the surface to be treated, effective formation of the plasma in the area of the surface to be treated and a large treatment depth are advantageously achieved.

The second length is in this case less than the first length or equal to the first length. The first length and the second length are accordingly selected such that the length of the bristle field (first length), which is predefined by the length of the longest bristles of the bristle field, is greater than the second length or equal to the second length to which the at least one electrode extends into the bristle field, such that the at least one electrode extending into the bristle field does not protrude beyond the bristle field.

In advantageous embodiments, the electrode arrangement of the plasma treatment device according to the invention may in particular have a plurality of electrodes that extend in the described manner into the bristle field in the direction of the contact surface. The different electrodes of the plurality of electrodes may in this case be electrically conductively connected to one another and/or connected to the same electrical potential. The different electrodes of the plurality of electrodes may however also be galvanically isolated from one another and/or connected to different electrical potentials.

The bristles of the bristle field may protrude at an angle, in particular approximately at right angles, from the base surface of the bristle carrier.

In one advantageous embodiment of the invention, the bristles of the bristle field may be made of a highly flexible dielectric plastic. Flexible silicones, in particular silicone rubbers, are for example suitable for this purpose. The bristles may however also be made of a different material. The bristles may in principle be made of any material suitable for this purpose, in a manner known per se.

In one advantageous development of the invention, in particular at least three electrodes or at least four electrodes or at least five electrodes or at least six electrodes or at least eight electrodes or at least ten electrodes or at least 15 electrodes or at least 20 electrodes or at least 25 electrodes or at least 30 electrodes or at least 40 electrodes or at least 50 electrodes may extend into the bristle field.

In one advantageous development of the invention, the plasma treatment device according to the invention may in particular not be designed for a treatment within the oral cavity of a living being with a dielectric barrier plasma. The plasma treatment device according to the invention may advantageously be designed in particular to treat a surface outside the oral cavity of a living being with a dielectric barrier plasma.

In a further advantageous development of the invention, the brush head of the plasma treatment device according to the invention might not be designed in a special shape for a special treatment.

In a further advantageous development of the invention, the plasma treatment device according to the invention may in particular be designed such that the brush head does not have a flat bottom section with a length and a width from which a plurality of webs extending over the width rise up over the length, said webs being designed for insertion into interdental spaces and the electrode arrangement extending in one piece into said webs.

In a further advantageous development of the invention, the plasma treatment device according to the invention may in particular be designed such that a plurality of webs do not rise up from the base surface of the bristle carrier, said webs being designed for insertion into interdental spaces and the electrode arrangement extending in one piece into said webs. In a further advantageous development of the invention, the plasma treatment device according to the invention may in particular be designed such that a plurality of webs do not rise up from the base surface of the bristle carrier, the electrode arrangement extending in one piece into said webs. In a further advantageous development of the invention, the plasma treatment device according to the invention may in particular be designed such that a plurality of webs do not rise up from the base surface of the bristle carrier, the electrode arrangement extending into said webs.

In a further advantageous development of the invention, provision is made for the at least one electrode of the electrode arrangement to extend into the intermediate spaces in the bristle field.

The at least one electrode extending into the bristle field may in this case be surrounded on all sides by the bristle field, in particular in a plane running substantially parallel to the contact surface. The plasma treatment device according to the invention may accordingly be designed such that the at least one electrode that extends into the bristle field does not extend to the edge of the bristle field in the plane running substantially parallel to the contact surface.

An electrode that extends into the bristle field in the sense of the present invention may accordingly be an electrode that extends into the intermediate spaces in the bristle field.

An electrode that extends into the bristle field in the sense of the present invention may in principle however also be an electrode that extends next to the bristles of the bristle field, in particular substantially parallel to the bristles of the bristle field, in the direction of the contact surface.

The at least one electrode extending into the bristle field is therefore not necessarily arranged such that it is surrounded by the bristles of the bristle field in the plane running substantially parallel to the contact surface, but it may also be arranged next to the bristles in this plane. In such an embodiment, it is also conceivable for example for the bristle field to be surrounded by the at least one electrode in the plane running substantially parallel to the contact surface. The bristle field may in this case be surrounded in particular on all sides by the at least one electrode, for example in a ring shape, in the plane running substantially parallel to the contact surface.

In a further advantageous development of the invention, provision is made for the at least one electrode of the electrode arrangement to extend into at least one bristle.

An electrode that extends into the bristle field in the sense of the present invention may thus also be an electrode that extends into a bristle of the bristle field.

In a further advantageous development of the invention, provision is made for at least some of the bristles to be formed partially or completely from the dielectric. All of the bristles may advantageously in particular be formed partially or completely from the dielectric. Some of the bristles or all of the bristles into which the at least one electrode of the electrode arrangement extends may advantageously in particular be formed partially or completely from the dielectric.

In a further advantageous development of the invention, provision is made for at least some of the bristles to be designed as hollow bristles, each of which has a cavity extending in the longitudinal direction of the bristle and into which an electrode of the electrode arrangement extends.

In a further advantageous development of the invention, provision is made for the at least one electrode of the electrode arrangement to consist of a castable plastic provided with conductive additives. The at least one electrode of the electrode arrangement that extends into at least one bristle may advantageously in this case in particular consist of a castable plastic provided with conductive additives.

The conductive additives create electrical conductivity in the plastic, such that it is able to be used to produce the electrode. The castable plastic may in this case in particular be a silicone. Suitable conductive additives are for example metal particles, carbon particles or the like.

Such an embodiment of the plasma treatment device according to the invention having an electrode made of a castable plastic provided with conductive additives is particularly advantageous, since it allows a material bond between the electrode and a dielectric that is likewise made of plastic and covers the electrode. The material bond in this case results from the plastics themselves, and does not require any additional adhesive layer at the boundary layer between the electrode and the dielectric. The electrode and the dielectric covering the electrode may thus be formed as a single material, as it were, and thus form a particularly durable connection. This is particularly important because the at least one electrode extending into the bristle field may be subjected to severe bending during the treatment.

This is particularly advantageous when the electrode of the electrode arrangement, consisting of a castable plastic provided with conductive additives, extends into at least one bristle that is formed partially or completely from the dielectric. This results in a particularly simple and therefore inexpensive-to-produce embodiment of the plasma treatment device according to the invention, in which the at least one electrode of the electrode arrangement extends into at least one bristle.

In a further advantageous development of the invention, provision is made for the at least one electrode of the electrode arrangement to consist of a conductive ceramic. The ceramic may in this case be provided with conductive additives. The conductive additives create electrical conductivity in the ceramic, such that it is able to be used to produce the electrode. Suitable conductive additives are for example metal particles, carbon particles or the like.

In a further advantageous development of the invention, provision is made for the electrode arrangement to be partially embedded in the bristle carrier, wherein the at least one electrode of the electrode arrangement protrudes from the bristle carrier in the direction of the contact surface. The part of the electrode protruding from the bristle carrier is in this case completely covered by the dielectric in the direction of the surface to be treated.

In a further advantageous development of the invention, provision is made for the bristle carrier to be formed partially or completely from the dielectric. The dielectric partially or completely forming the bristle carrier may advantageously be a flexible dielectric. The dielectric partially or completely forming the bristle carrier may in particular be made of silicone, in particular of silicone rubber. The dielectric partially or completely forming the bristle carrier may however also for example be made of a rigid plastic.

In a further advantageous development of the invention, provision is made for the at least one electrode to be surrounded on all sides by the bristle field in a plane running substantially parallel to the contact surface. The brush head may in particular have a bristle field designed as a bristle ring, wherein the bristle ring surrounds the at least one electrode on all sides in the plane running substantially parallel to the contact surface.

In a further advantageous development of the invention, provision is made for the bristle carrier to have a through-opening through which the at least one electrode of the electrode arrangement extends. The electrode extending through the through-opening may in this case be surrounded on all sides by the bristle field in a plane running substantially parallel to the contact surface. The brush head may in this case in particular have a bristle field designed as a bristle ring, wherein the bristle ring surrounds the electrode extending through the through-opening on all sides in the plane running substantially parallel to the contact surface.

Such an embodiment of the plasma treatment device according to the invention, in which the bristle carrier has a through-opening through which the electrode extends, offers the advantage that it is possible to implement a simple structural solution in which the bristle carrier and the bristle field are able to be detached for cleaning or exchange of the plasma treatment device, while the electrode, which extends through the through-opening of the bristle carrier in the assembled state, remains on the device.

In a further advantageous development of the invention, provision is made for the at least one electrode of the electrode arrangement to be flexible. For this purpose, the at least one electrode of the electrode arrangement may consist of a flexible and electrically conductive material. The flexibility may in this case result from an elastic deformability, but also from a plastic deformability of the material of the electrode. For this purpose, the at least one flexible electrode of the electrode arrangement may be made for example of a conductive and flexible plastic, a conductive plastic powder, conductive plastic granules, a metal powder and/or metal granules. Electrically conductive silicone may in particular serve as electrically conductive plastic. The electrical conductivity of the plastic may in this case be generated in the manner described above by virtue of providing the plastic with conductive additives.

Such an embodiment of the plasma treatment device according to the invention, in which the at least one electrode is flexible, offers the advantage that not only are the flexible bristles of the bristle field able to adapt their shape to the surface to be treated, but also the electrode is able to adapt its shape to the surface to be treated during the treatment. This achieves a particularly high level of effectiveness both for the plasma treatment and for the mechanical treatment.

In a further advantageous embodiment of the invention, the at least one electrode of the electrode arrangement may however also be rigid.

It is of course also possible for the electrode arrangement to have a plurality of electrodes and to comprise both flexible and rigid electrodes. The electrode arrangement may accordingly have at least one flexible electrode and/or at least one rigid electrode.

In a further advantageous development of the invention, provision is made for the bristle carrier to have a circular or elliptical or ring-shaped cross section. This offers both the advantage of easy handling and the advantage that this shape of the bristle carrier is well-suited for designing the bristle carrier to be movable, in particular movable in rotation.

In a further advantageous development of the invention, provision is made for the electrode arrangement to have a plurality of finger-shaped electrodes that are surrounded by a dielectric layer and protrude from the base surface of the bristle carrier into the bristle field. The finger-shaped electrodes and the dielectric layer surrounding them may in particular in this case be designed to be flexible.

The plasma treatment device may accordingly have a plurality of electrode fingers that are each formed from a finger-shaped electrode and a dielectric layer surrounding the finger-shaped electrode. The electrode fingers in this case protrude from the base surface of the bristle carrier in the direction of the contact surface. The electrode fingers may in this case extend into the intermediate spaces in the bristle field.

The cross section of the individual finger-shaped electrodes and/or of the individual electrode fingers may in this case in particular be substantially circular and/or elliptical and/or substantially square. The cross section of the individual finger-shaped electrodes and/or of the individual electrode fingers may advantageously have a longest and a shortest side, wherein the longest side has a length that is no more than two and a half times, in particular no more than twice, in particular no more than one and a half times the length of the shortest side.

The electrode fingers may advantageously in particular be designed to be thin and flexible enough that they essentially have the appearance and the mechanical properties of bristles, in particular bristles of a conventional brush.

Such an embodiment of the plasma treatment device according to the invention, which has a plurality of finger-shaped electrodes surrounded by a dielectric layer, offers the advantage that a multiplicity of electrodes extending into the bristle field are able to be distributed within the bristle field. This makes it possible to achieve a particularly homogeneous density of the bristles within the bristle field, which enables a particularly effective mechanical treatment of the surface to be treated.

In a further advantageous development of the invention, provision is made for the at least one electrode of the electrode arrangement extending into the bristle field to have a flat end face facing the contact surface and that is covered by a flat end wall of the dielectric in the direction of the contact surface.

Such an embodiment of the plasma treatment device according to the invention having an electrode that has a flat end face facing the contact surface offers the advantage that particularly effective and uniform formation of the plasma between the flat end wall, covering the flat end face, of the dielectric and the surface to be treated is thereby achieved.

In a further advantageous development of the invention, provision is made for the flat end wall of the dielectric to have at least one spacer on its side facing the contact surface. The flat end wall of the dielectric may advantageously have a plurality of spacers on its side facing the contact surface. The at least one spacer may in this case advantageously be formed from the dielectric. The at least one spacer may in particular be formed in one piece with the flat end wall of the dielectric.

Such an embodiment of the plasma treatment device according to the invention having at least one spacer on the flat end wall of the dielectric offers the advantage of being able to ensure a defined distance between the flat end wall of the dielectric and the surface to be treated during the treatment. This ensures the presence of an intermediate space, in which the plasma is able to form, between the flat end wall and the surface to be treated during the treatment.

In a further advantageous development of the invention, provision is made for some or all of the bristles of the bristle field on the side of the bristle field facing the contact surface to protrude beyond the at least one electrode of the electrode arrangement extending into the bristle field and the dielectric covering the electrode.

The bristles protruding beyond the electrode advantageously ensure that a distance between the surface to be treated and that end of the at least one electrode facing the surface to be treated is maintained during the treatment when the ends of the bristles rest on the surface to be treated. In the resultant intermediate space between the at least one electrode and the surface to be treated, the plasma is able to form in the immediate vicinity of the surface to be treated, so as to achieve a particularly effective plasma treatment.

In a further advantageous development of the invention, provision is made for the second length to be at least 40% or at least 50% or at least 60% or at least 70% or at least 80% or at least 90% or at least 95% or at least 99% of the first length.

Since a larger ratio of the second length to the first length is synonymous with an electrode extending further into the bristle field, a second length selected to be greater in relation to the first length offers the advantage that the electrode extending into the bristle field has a smaller distance from the surface to be treated during the treatment when the ends of the bristles of the bristle field rest on the surface to be treated. The effectiveness of the plasma formation in the area of the surface to be treated may thereby be improved, since a greater distance between the electrode and the surface to be treated complicates the plasma formation.

In a further advantageous development of the invention, provision is made for the bristle carrier to mounted so as to be movable and for the treatment device to have a drive unit that is configured to drive a movement of the bristle carrier. The bristle carrier may in this case advantageously in particular be mounted so as to be movable in rotation. The drive unit may in this case in particular be configured to drive a rotational movement and/or a rotationally oscillating movement of the bristle carrier. The drive unit may in particular be an electric drive unit.

Such a development of the invention offers the advantage that the effectiveness of the mechanical treatment of the surface to be treated is improved, and the handling of the plasma treatment device during the treatment is able to be simplified.

In a further advantageous development of the invention, provision is made for the electrode arrangement to comprise a plurality of electrodes and to have at least one electrically conductive distributor plate. The distributor plate is in this case connected to the high-voltage supply line and a plurality of electrodes and is configured to distribute the high-voltage signal to the electrodes connected to the distributor plate.

Such an embodiment of the plasma treatment device according to the invention having a distributor plate offers the advantage of a low-loss and at the same time structurally easy-to-implement distribution of the high-voltage signal to the plurality of electrodes of the electrode arrangement.

In a further advantageous development of the invention, provision is made for the line arrangement to comprise a plurality of high-voltage supply lines to which different high-voltage signals are able to be applied, and for the electrode arrangement to comprise a plurality of electrodes that form a plurality of electrode groups that are electrically insulated from one another. The different electrode groups are in this case connected to different high-voltage supply lines and are able to be supplied with different high-voltage signals.

It is therefore proposed for the electrode arrangement to have a plurality of electrode groups that are able to be supplied with different high-voltage signals. In the simplest case, the electrode arrangement may in this case have two electrodes that are electrically insulated from one another and each form an electrode group consisting of just one electrode.

The different electrode groups may advantageously in this case be supplied with different high-voltage signals that are of opposing polarity to one another. The different high-voltage signals may in this case in particular be of opposing polarity and the same absolute value. The high-voltage signals may advantageously in particular be in the form of AC high-voltage signals, and the different electrode groups may be supplied with AC high-voltage signals in phase opposition. The AC high-voltage signals in phase opposition may in this case in particular have a substantially identical peak value.

Such an embodiment offers the advantage that the resulting electric fields are destructively superimposed in their area of overlap and cancel one another out at some distance from the electrodes, such that the electric field required for the plasma formation remains limited to the close area relevant to the plasma treatment. This furthermore offers the advantage of being able to avoid undesired field peaks, which could result from constructive superimposition of the electric fields generated by the different electrode groups in their area of overlap.

In a further advantageous development of the invention, provision is made for the electrode arrangement to have a plurality of distributor plates that are electrically insulated from one another, wherein the electrodes of the different electrode groups are connected to different distributor plates and the different distributor plates are connected to different high-voltage supply lines.

There is thus provision for the electrode arrangement to have a plurality of electrically conductive distributor plates that are electrically insulated from one another, wherein each distributor plate is connected to a plurality of electrodes and to one of the high-voltage supply lines and is configured to distribute the high-voltage signal to the electrodes connected to the distributor plate, and wherein the electrodes of the different electrode groups are connected to different distributor plates and the different distributor plates are connected to different high-voltage supply lines.

Such a development of the plasma treatment device according to the invention having a plurality of distributor plates electrically insulated from one another offers the advantage that it enables low-loss and at the same time easy-to-implement distribution of different high-voltage signals to different electrode groups.

In a further advantageous development of the invention, provision is made for the different distributor plates of the plurality of distributor plates that are electrically insulated from one another to be arranged in the same plane.

In a further advantageous development of the invention, provision is made for at least one of the distributor plates to be cross-sectionally surrounded by another distributor plate. This offers the advantage of a simple structural implementation of a plurality of distributor plates, which is also suitable for an embodiment of the plasma treatment device according to the invention having a bristle carrier that is mounted and driven so as to be movable in rotation.

In a further advantageous development of the invention, provision is made for a distributor plate having a circular cross section to be surrounded by at least one other distributor plate having a circular cross section. As an alternative or in addition thereto, a plurality of distributor plates having a circular cross section and different diameters may be arranged concentrically.

The different distributor plates may also be arranged in different planes. The different distributor plates may in this case in particular be arranged one behind the other as seen from the contact surface.

Furthermore, at least one distributor plate may have at least one through-hole through which at least one electrode connected to another distributor plate extends. This offers the advantage of a simple structural implementation of a plurality of distributor plates that are arranged in different planes in the manner described above.

In a further advantageous development of the invention, provision is made for the plasma treatment device to have a handle part and for at least one removable part of the brush head, comprising the bristle carrier and the bristle field, to be connected to the handle part in a detachable and exchangeable manner by way of a mechanical connection arrangement.

It is also conceivable in this case for the entire brush head to be designed as a removable brush head that is connected to the handle part in a detachable and exchangeable manner by way of a mechanical connection arrangement.

Such an embodiment of the plasma treatment device according to the invention having a removable brush head or at least a removable part of the brush head that comprises the bristle carrier and the bristle field offers the advantage that the part of the brush head comprising the bristle carrier and the bristle field is able to be removed from the plasma treatment device following the treatment and replaced with an unused specimen. The part of the brush head comprising the bristle carrier and the bristle field may thereby advantageously be designed as a disposable article. This embodiment furthermore offers the advantage that the cleaning, required following the treatment, of the part of the brush head comprising the bristle carrier and the bristle field is considerably simplified. Compliance with high hygiene standards may thereby be ensured.

In a further advantageous development of the invention, provision is made for the removable part of the brush head to comprise part of the electrode arrangement or the entire electrode arrangement. The handle part in this case has a contact arrangement and the removable part of the brush head has a connector connected to the electrode arrangement and that makes contact with the contact arrangement of the handle part when the removable part of the brush head is connected to the handle part by way of the mechanical connection arrangement.

Such an embodiment of the plasma treatment device according to the invention offers the advantage for example that a removable brush head or a removable part of the brush head with the associated advantages explained above may also be implemented when the electrode arrangement is at least partially embedded in the bristle carrier.

In a further advantageous development of the plasma treatment device according to the invention, provision is made for the handle part to contain all of the stages required to generate the high-voltage signal.

On the one hand, this offers the advantage that no high voltage has to be carried outside the device, such that it is significantly easier to guarantee the required safety of the device. On the other hand, this offers the advantage that the comparatively complex and therefore expensive components for generating the high-voltage signal remain in the plasma treatment device and do not have to be exchanged when the removable part of the brush head or the entire brush head is exchanged. As a result, the part of the brush head comprising the bristle carrier and the bristle field or the entire brush head may be produced inexpensively and even implemented as a disposable item.

The object mentioned at the outset is furthermore achieved according to the invention by a brush head of a plasma treatment device of the type described above.

The object mentioned at the outset is furthermore achieved by a removable part of a brush head of a plasma treatment device of the type described above.

The invention is intended to be explained in more detail below with reference to the exemplary embodiments illustrated schematically in the accompanying drawings. In the figures:

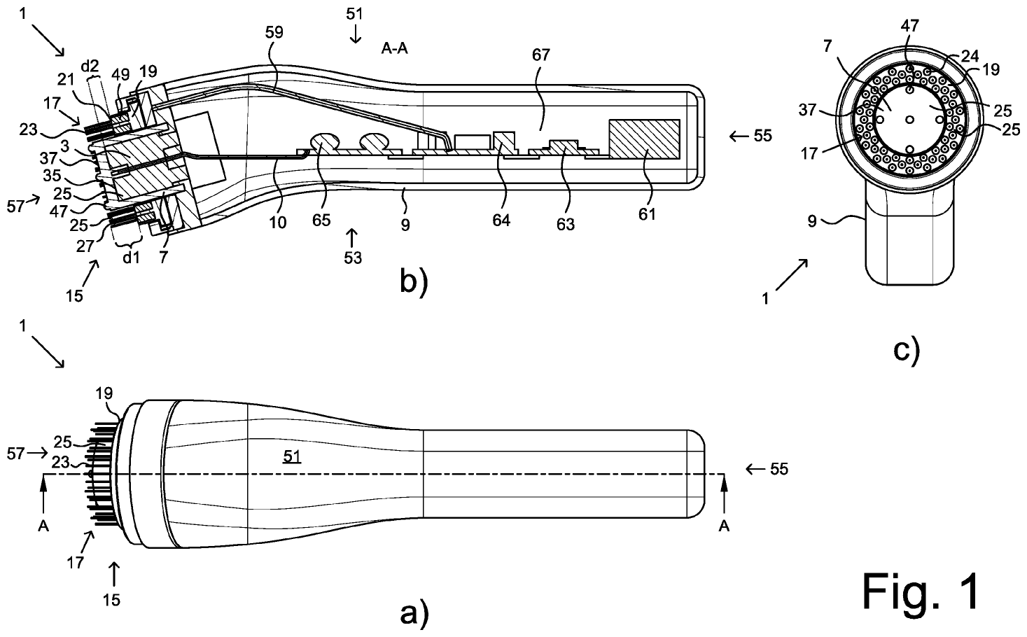

FIG. 1a) shows a view of an upper side of a first embodiment of a plasma treatment device according to the invention;

FIG. 1b) shows a longitudinal section along the line A-A in FIG. 1a);

FIG. 1c) shows a view of a contact side of the first embodiment of the plasma treatment device;

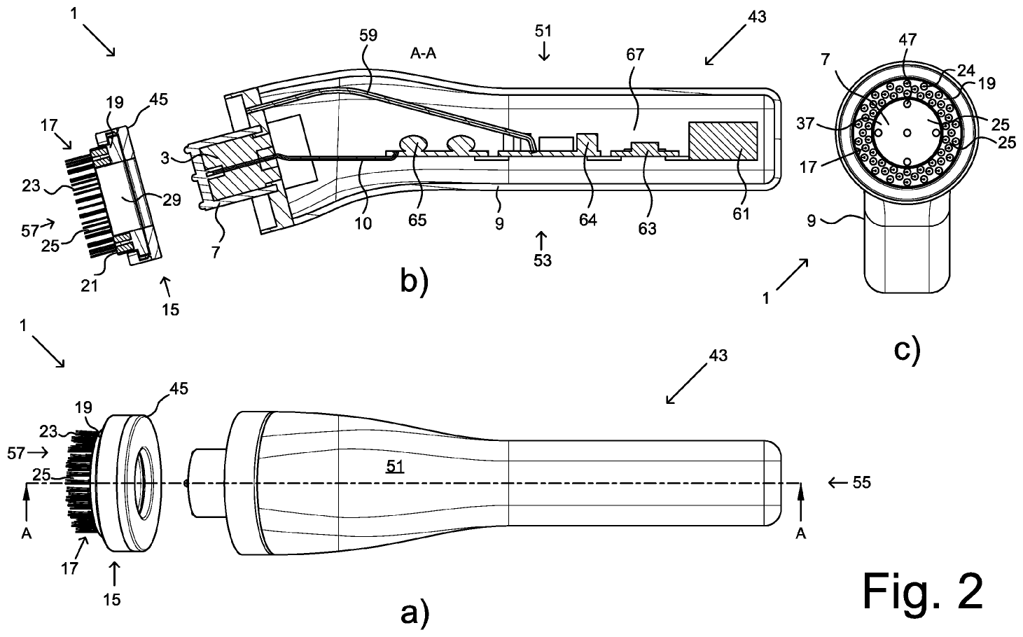

FIG. 2a) shows a view of an upper side of the first embodiment of the plasma treatment device with the brush head removed;

FIG. 2b) shows a longitudinal section along the line A-A in FIG. 2a);

FIG. 2c) shows a view of the contact side of the first embodiment of the plasma treatment device;

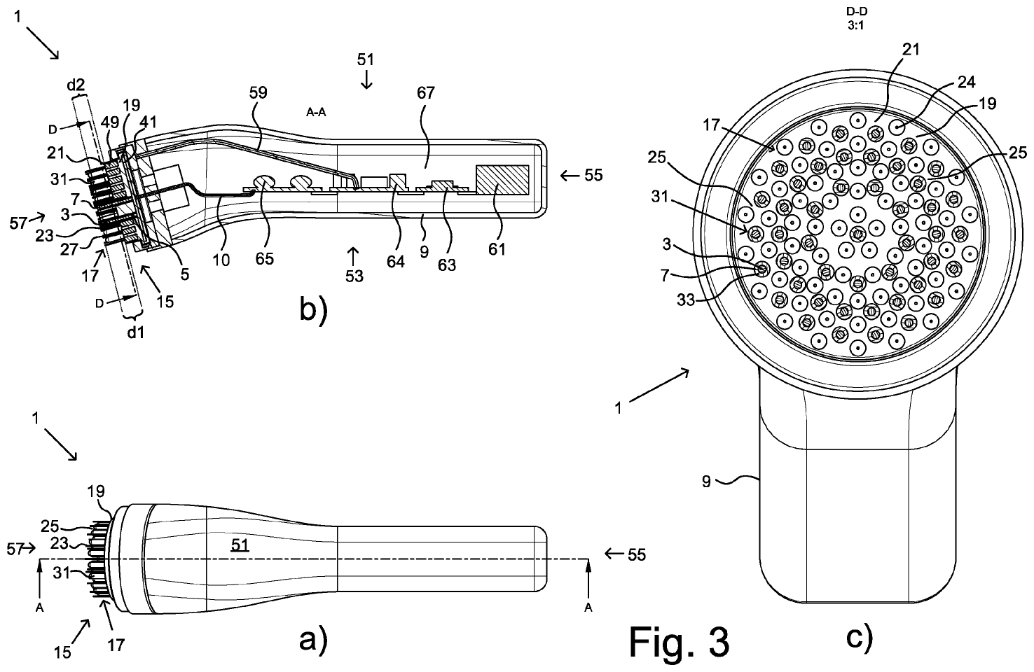

FIG. 3a) shows a view of an upper side of a second embodiment of a plasma treatment device according to the invention;

FIG. 3b) shows a longitudinal section along the line A-A in FIG. 3a);

FIG. 3c) shows a cross section along the line D-D in FIG. 3b);

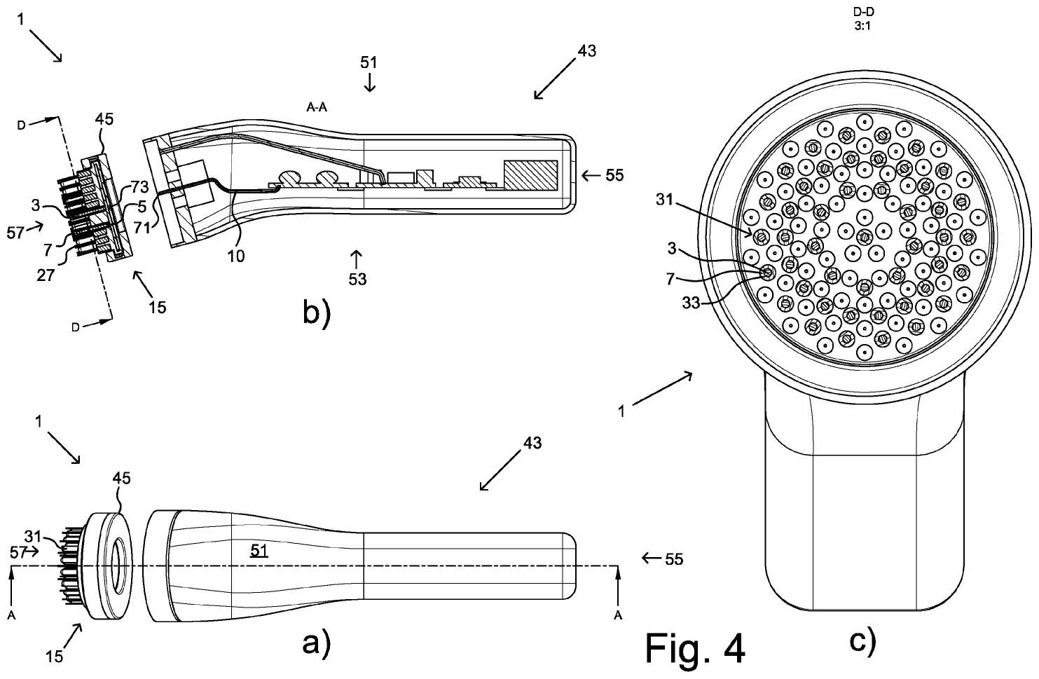

FIG. 4a) shows a view of an upper side of the second embodiment of the plasma treatment device with the brush head removed;

FIG. 4b) shows a longitudinal section along the line A-A in FIG. 4a);

FIG. 4c) shows a cross section along the line D-D in FIG. 4b);

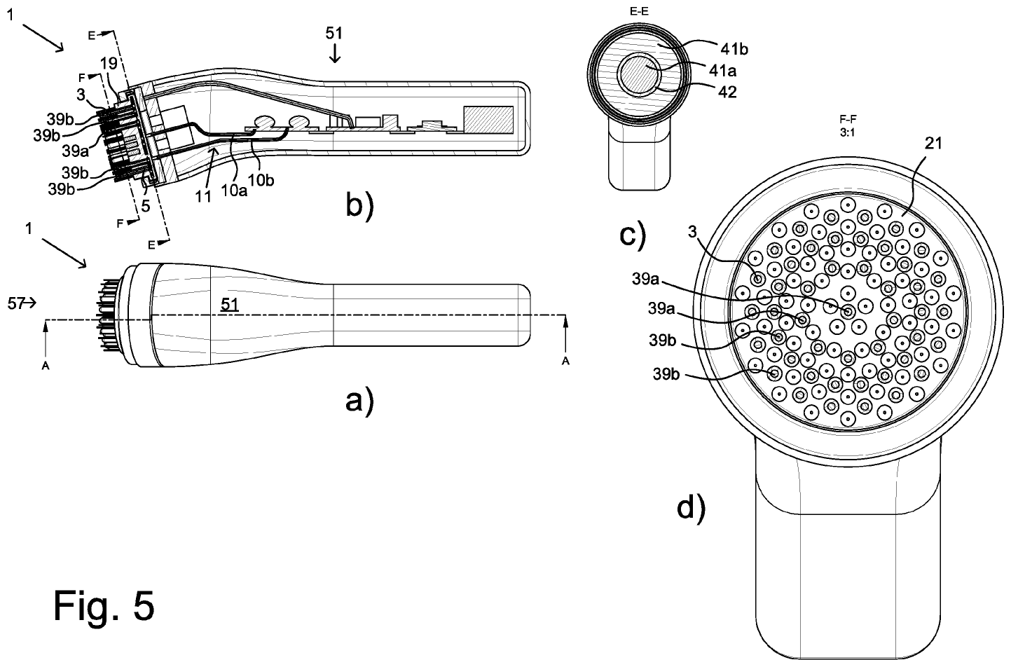

FIG. 5a) shows a view of an upper side of a third embodiment of a plasma treatment device according to the invention;

FIG. 5b) shows a longitudinal section along the line A-A in FIG. 5a);

FIG. 5c) shows a cross section along the line E-E in FIG. 5b);

FIG. 5d) shows a cross section along the line F-F in FIG. 5b);

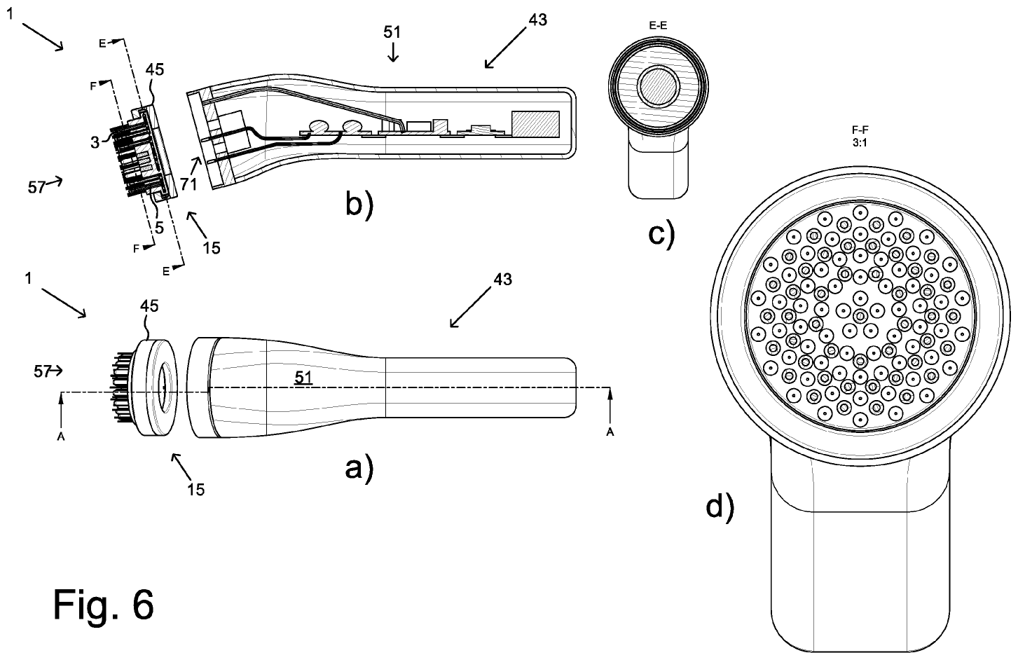

FIG. 6a) shows a view of an upper side of the third embodiment of the plasma treatment device with the brush head removed;

FIG. 6b) shows a longitudinal section along the line A-A in FIG. 6a);

FIG. 6c) shows a cross section along the line E-E in FIG. 6b);

FIG. 6d) shows a cross section along the line F-F in FIG. 6b);

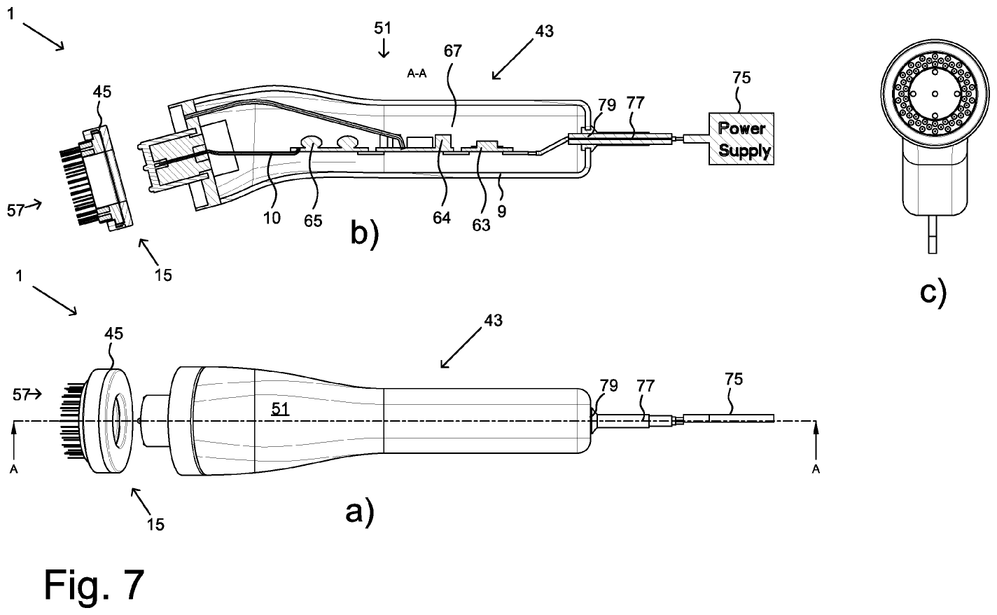

FIG. 7a) shows a view of an upper side of a fourth embodiment of a plasma treatment device according to the invention with the brush head removed;

FIG. 7b) shows a longitudinal section along the line A-A in FIG. 7a);

FIG. 7c) shows a view of a contact side of the fourth embodiment of the plasma treatment device;

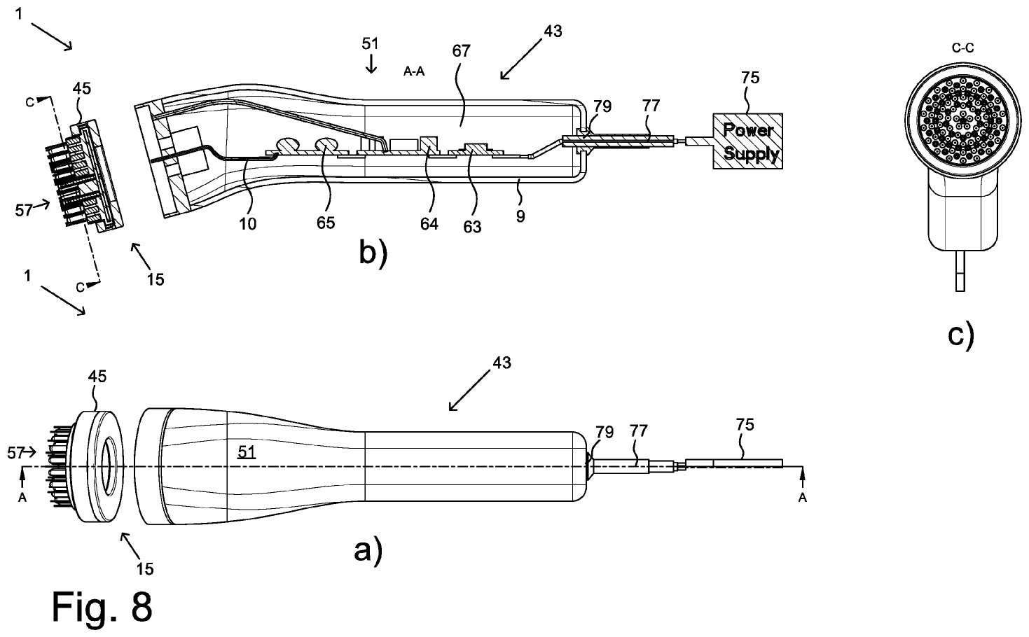

FIG. 8a) shows a view of an upper side of a fifth embodiment of a plasma treatment device according to the invention with the brush head removed;

FIG. 8b) shows a longitudinal section along the line A-A in FIG. 8a);

FIG. 8c) shows a cross section along the line C-C in FIG. 8b);

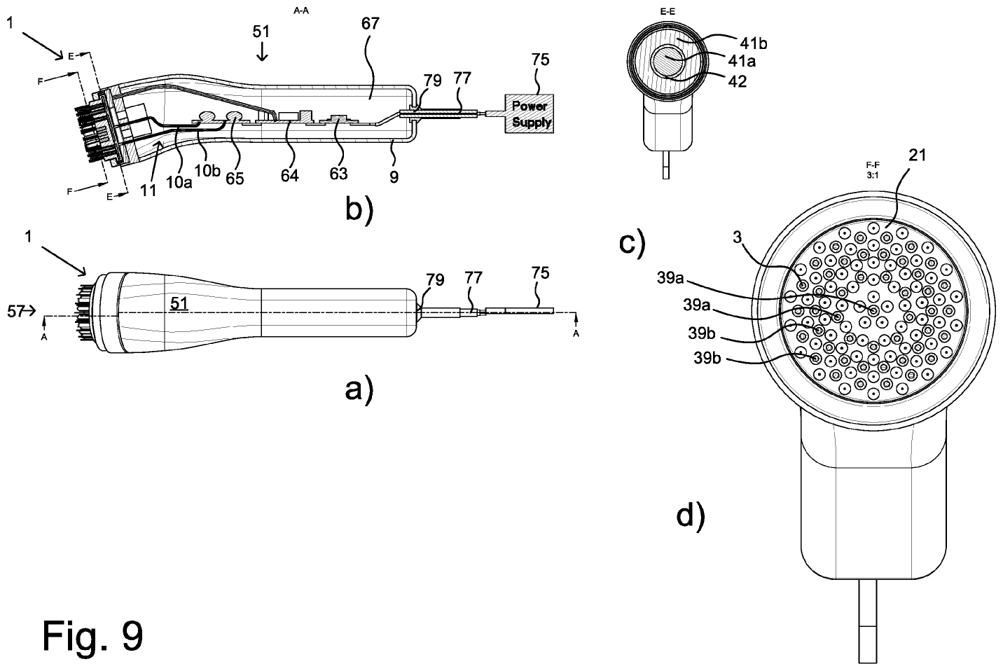

FIG. 9a) shows a view of an upper side of a sixth embodiment of a plasma treatment device according to the invention;

FIG. 9b) shows a longitudinal section along the line A-A in FIG. 9a);

FIG. 9c) shows a cross section along the line E-E in FIG. 9b);

FIG. 9d) shows a cross section along the line F-F in FIG. 9b);

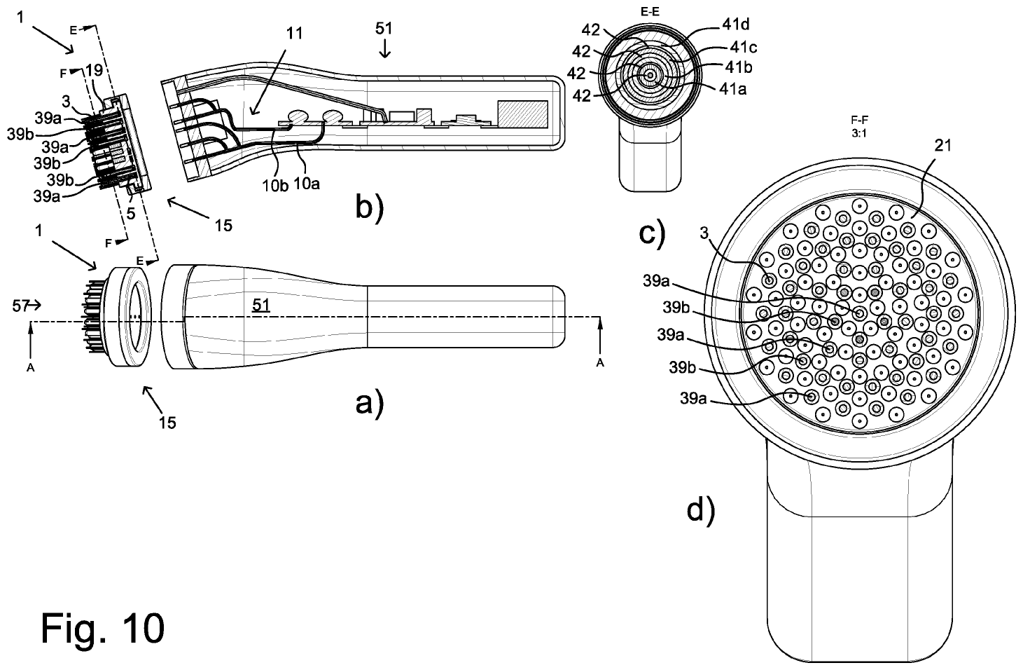

FIG. 10a) shows a view of an upper side of a seventh embodiment of a plasma treatment device according to the invention with the brush head removed;

FIG. 10b) shows a longitudinal section along the line A-A in FIG. 10a);

FIG. 10c) shows a cross section along the line E-E in FIG. 10b);

FIG. 10d) shows a cross section along the line F-F in FIG. 10b);

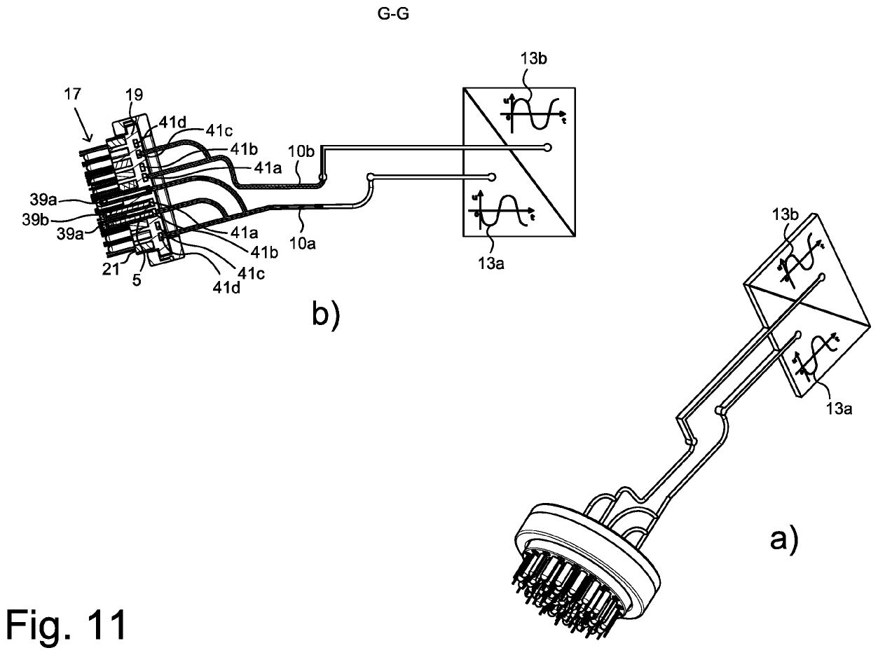

FIG. 11a) shows a perspective view of components of the seventh embodiment of the plasma treatment device having two electrode groups that are supplied with AC high-voltage signals in phase opposition;

FIG. 11b) shows a longitudinal section of the components illustrated in FIG. 11a);

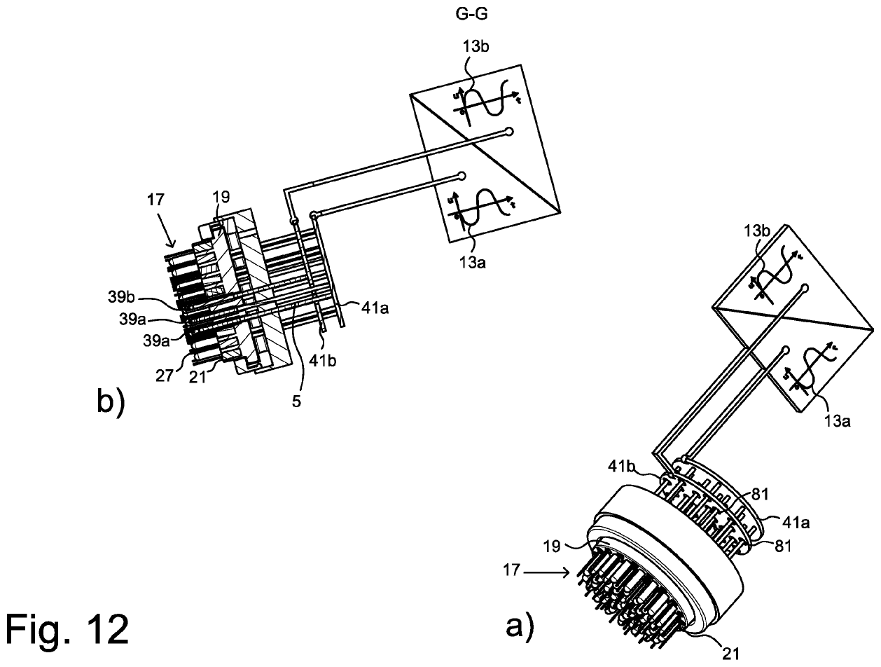

FIG. 12a) shows a perspective view of components of an eighth embodiment of a plasma treatment device according to the invention having two electrode groups that are supplied with AC high-voltage signals in phase opposition;

FIG. 12b) shows a longitudinal section of the components illustrated in FIG. 12a).

In the figures, the same reference signs are used for elements that correspond to one another.

The exemplary embodiment shown in FIG. 1a) shows a view of an upper side 51 of a first embodiment of a plasma treatment device 1 according to the invention, which is designed to treat a surface with a dielectric barrier plasma. Further details of the structure of this first embodiment may be derived from the longitudinal section illustrated in FIG. 1b) and the view, illustrated in FIG. 1c), of a contact side 57 of the plasma treatment device 1.

It may be seen in FIGS. 1a) and 1b) that the plasma treatment device, in addition to the upper side 51 and the contact side 57, has a lower side 53 and a rear side 55. It may also be seen that the plasma treatment device, in this first embodiment, has an electrode arrangement having a single electrode 3 that is completely covered by a dielectric 7 in the direction of the surface to be treated. The dielectric 7 thus completely covers the electrode 3, in particular in the direction of the contact side 57. The plasma treatment device furthermore has a housing 9 that contains a line arrangement having a high-voltage supply line 10. The electrode 3 is connected to the line arrangement and is able to be supplied, via the high-voltage supply line 10, with a high-voltage signal able to be applied to the high-voltage supply line 10.

It may also be seen that the plasma treatment device 1 has a brush head 15. The brush head 15 has a bristle field 17 having a multiplicity of flexible bristles 23 and having intermediate spaces 25 between the bristles 23. The bristles 23 are in this case arranged in bristle bundles 24 that each comprise a plurality of bristles 23. The bristle field 17 thus has a multiplicity of bristle bundles 24 formed from bristles 23. The brush head 15 furthermore has a bristle carrier 19 that has a base surface 21 from which the bristles 23 of the bristle field 17 protrude in the direction of a contact surface 27. The contact surface 27 is in this case defined by the ends of the longest bristles 23 of the bristle field 17 that face away from the base surface 21. The bristle carrier 19 also has anchoring areas 49 in which the bristles 23 are anchored. In the exemplary embodiment that is shown, a respective bristle bundle 24 is in this case anchored in each anchoring area 49.

It may furthermore be seen in FIG. 1b) that the bristle field 17 has a first length d1 defined by the distance between the base surface 21 and the contact surface 27, and the electrode 3 extends from the base surface 21 into the bristle field 17 with a second length d2 in the direction of the contact surface 27. The second length d2 is in this case less than the first length d1 and, in this first embodiment, is approximately 50% of the first length d1.

The bristle field 17 has a multiplicity of smaller intermediate spaces 25 between the bristles 23. As becomes clear in particular in FIG. 1c), the bristle field 17 in this first embodiment furthermore has a larger intermediate space 25 with a circular cross section, which is surrounded by the bristles 23 of the bristle field 17 and into which the electrode 3 extends. The bristles 23 of the bristle field 17 in this case form a bristle ring that surrounds the electrode 3. In the exemplary embodiment that is shown, the electrode 3 is in this case cross-sectionally surrounded on all sides by the bristle ring formed from the bristles 23 of the bristle field 17.

In the first embodiment of the plasma treatment device according to the invention shown in FIG. 1, the electrode 3 extending into the bristle field 17 is designed as a solid, cylindrical electrode that is enclosed by a pot-shaped dielectric 7. The electrode 3 has a flat end face 35 facing the contact surface 27 and that is covered in the direction of the contact surface 27 by a flat end wall 37, having a circular cross section, of the dielectric 7. The flat end wall 37 of the dielectric 7 thus covers the flat end face 35 of the electrode 3 in the direction of the contact side 57. The flat end wall 37 of the dielectric 7 has a total of five spacers 47 on its side facing the contact surface 27, the arrangement of which spacers may be seen in FIG. 1c). The spacers 47 are formed from the dielectric 7 and formed in one piece with the flat end wall 37 of the dielectric 7.

In the exemplary embodiment shown in FIG. 1, as in all of the exemplary embodiments shown in the following figures, the electrode arrangement of the plasma treatment device 1 is configured such that the surface to be treated is used as counter-electrode.

It may also be seen in the longitudinal section illustrated in FIG. 1b) that the plasma treatment device 1 in this first embodiment has a battery 61 that provides the required supply voltage and is designed as a rechargeable battery (accumulator). The battery 61 is in this case arranged in the interior 67 of the housing 9, that is to say the housing 9 contains the battery 61. It may also be seen that the plasma treatment device 1 has a high-voltage stage 65 that is arranged in the housing 9 and that is connected at output to the high-voltage supply line 10 of the line arrangement. The housing 9 furthermore contains an electronic controller 63 and an intermediate stage 64, by way of which an AC voltage signal is generated from a DC voltage supplied by the battery 61, which AC voltage signal is converted into a high-voltage signal in the form of an AC high-voltage signal by way of the high-voltage stage 65.

For this purpose, the electronic controller 63 controls the intermediate stage 64 by way of which an AC voltage having an increased peak voltage is generated from the DC voltage of the battery 61 in a manner known per se, this peak voltage being able to be for example between 50 V and 500 V. The intermediate stage 64, via its output, feeds the high-voltage stage 65, in which for example high-voltage pulses of 13 kV to 15 kV are generated.

The electrode 3 may thereby be supplied with the AC high-voltage signal via the high-voltage supply line 10. In this exemplary embodiment, the plasma treatment device 1 is thus designed to generate the high voltage required for the plasma generation itself.

It may be seen from FIG. 1c) that the bristle carrier 19 of this first embodiment of the plasma treatment device 1 has a circular cross section. The bristles 23 bundled into bristle bundles 24 are arranged in a manner distributed over the cross section of the bristle carrier 19 and form a bristle ring that surrounds the electrode 3, which has a circular cross section and is covered by the dielectric 7.

In this first embodiment of the plasma treatment device 1, the bristle carrier 19 is also designed to be movable in rotation. For this purpose, the bristle carrier 19 is mounted so as to be movable in rotation and has an electric drive unit (not shown in FIG. 1) that is configured to drive a rotationally oscillating movement of the bristle carrier. A control line 59 is arranged in the interior 67 of the housing 9 for this purpose and serves to actuate the electric drive unit.

FIG. 2a) shows a view of an upper side of the first embodiment of the plasma treatment device 1 with the brush head 15 removed. FIG. 2b) in this respect shows a longitudinal section along the line A-A in FIG. 2a), and FIG. 2c) shows a view of the contact side 57 of the plasma treatment device 1.

It once again becomes clear in this case that, in this embodiment, the electrode 3, when the brush head 15 is not removed, extends into an intermediate space 25, having a circular cross section, of the bristle field 17 that is surrounded by the bristles 23. It may also be seen in the illustration of FIGS. 2a) and 2b) that the bristle carrier 19, in this first embodiment, has a through-opening 29 through which the cylindrical electrode 3 extends when the brush head 15 is not removed.

FIGS. 2a) and 2b) furthermore show that the plasma treatment device 1 has a handle part 43 and that the brush head 15 comprising the bristle carrier 19 and the bristle field 17 is connected to the handle part 43 in a detachable and exchangeable manner by way of a mechanical connection arrangement. The removable part 45 of the brush head 15, comprising at least the bristle carrier 19 and the bristle field 17, is thus formed in this exemplary embodiment by the entire brush head 15. The mechanical connection between the removable part 45 of the brush head 15 and the handle part 43 is in this case preferably designed as a snap-in connection, but may also be designed as a screw connection, bayonet connection or another mechanical connection.

In the first embodiment shown in FIGS. 2a) and 2b), the electrode 3 is fixedly connected to the handle part 43 of the plasma treatment device 1 and remains on the handle part 43 when the removable part 45 of the brush head 15 is removed from the handle part 43. The electrode 3, which is fixedly connected to the handle part 43, may extend into the bristle field 17 through the through-opening 29 in the bristle carrier 19 after the removable part 45 of the brush head 15 has been reconnected to the handle part 43.

As may be seen in FIG. 2b), the handle part of the plasma treatment device contains all of the stages required to generate the high-voltage signal. In this exemplary embodiment, these are the electronic controller 63, the intermediate stage 64 and the high-voltage stage 65.

FIG. 3a) shows a view of an upper side of a second embodiment of a plasma treatment device 1 according to the invention. Further details of the structure of this second embodiment may be derived from the longitudinal section illustrated in FIG. 3b) and the cross section, illustrated in FIG. 3c), of the plasma treatment device 1.

It may be seen in this case that the electrode arrangement 5 in this second embodiment, in contrast to the first embodiment explained above, has a plurality of electrodes 3. The electrodes 3 are in this case designed as finger-shaped electrodes that are surrounded by a dielectric layer 33 and extend from the base surface 21 of the bristle carrier 19 into the bristle field 17. In this case, a finger-shaped electrode 3 and an associated dielectric layer 33 surrounding the finger-shaped electrode 3 each form an electrode finger 31. In this second embodiment, the plasma treatment device accordingly has a plurality of electrode fingers 31 that protrude from the base surface 21 of the bristle carrier 19 in the direction of the contact surface 27. The electrode fingers 31 in this case extend into the intermediate spaces 25 in the bristle field 17. In this exemplary embodiment, the finger-shaped electrodes 3, like the dielectric layer 33 surrounding them, are designed to be flexible. For this purpose, the dielectric layer 33 is made of a silicone rubber. The finger-shaped electrodes 3 of the electrode arrangement 5 consist of a castable plastic provided with conductive additives, specifically of silicone, which is provided with conductive particles.

It may furthermore be seen in the longitudinal section illustrated in FIG. 3b) that the bristle field 17, in the same way as the first embodiment explained above, has a first length d1 defined by the distance between the base surface 21 and the contact surface 27, and the electrode 3 extends from the base surface 21 with a second length d2 into the bristle field 17 in the direction of the contact surface 27. The second length d2 is in this case less than the first length d1 and, in this second embodiment, is approximately 75% of the first length d1.

It may also be seen in FIG. 3b) that the electrode arrangement 5, in addition to the plurality of electrodes 3, has an electrically conductive distributor plate 41 that is connected to the high-voltage supply line 10 and all of the electrodes 3. The distributor plate 41 is configured to distribute the high-voltage signal, able to be applied to the high-voltage supply line 10, to the electrodes 3 connected to the distributor plate 41.

It may furthermore be seen in FIG. 3b) that the electrode arrangement 5 is partially embedded in the bristle carrier 19, specifically with some of the finger-shaped electrodes 3 and with the distributor plate 41, wherein the electrodes 3 of the electrode arrangement 5 protrude from the bristle carrier 19 in the direction of the contact surface 27. The bristle carrier 19 is formed from the dielectric 7 in this exemplary embodiment.

It may furthermore be seen from FIGS. 3a) and 3b) that, in the second embodiment shown there, the bristles 23 of the bristle field 17 on the side of the bristle field 17 that faces the contact surface 27, that is to say on the contact side 57 of the plasma treatment device, protrude beyond the electrode fingers 31 extending into the bristle field 17. The bristles 23, on the side of the bristle field 17 that faces the contact surface 27, thus protrude beyond the electrodes 3 extending into the bristle field 17 and the dielectric 7 covering the electrodes 3.

It may be seen from the illustration in FIG. 3c) that the bristle carrier 19, in this second embodiment, has a circular cross section. It may also be seen in FIG. 3c) that the electrode fingers 31, that is to say the finger-shaped electrodes 3 and the dielectric layers 33 surrounding them, are arranged in a manner distributed over the base surface 21 of the bristle carrier 19. An electrode 3 is in this case arranged centrally on the base surface 21 of the bristle carrier 19. A plurality of electrodes 3 are furthermore arranged on concentric circles around a center point of the circular cross section of the bristle carrier 19.

FIGS. 4a), 4b) and 4c) respectively show a view of an upper side 51, a longitudinal section and a cross section of the second embodiment of the plasma treatment device with the brush head 15 removed. It may be seen in these illustrations that the second embodiment shown here, in the same way as the first embodiment of the plasma treatment device 1 according to the invention discussed above, has a brush head 15 that is connected to a handle part 43 in a detachable and exchangeable manner by way of a mechanical connection arrangement. In contrast to the first embodiment explained above, in the second embodiment shown here, as is clear from the longitudinal section shown in FIG. 4b), provision is made for the removable part 45 of the brush head 15 to comprise at least part of the electrode arrangement 5, in the exemplary embodiment shown here specifically even the entire electrode arrangement.

In order to allow electrical contact between the removable part 45 of the brush head 15, which is in this case formed by the entire brush head 15, and the handle part 43, the removable part 45 of the brush head 15 has a connector 73 connected to the electrode arrangement 5 and that is designed as a connection pin in this exemplary embodiment, and the handle part 43 has a contact arrangement 71 that is designed as a connection socket in this exemplary embodiment. The contact arrangement 71 and the connector 73 are in this case designed such that, in the mounted state of the removable part 45 of the brush head 15, that is to say when the removable part 45 of the brush head 15 is connected to the handle part 43 by way of the mechanical connection arrangement, the connector 73 makes electrical contact with the contact arrangement 71.

For the rest, with regard to the second embodiment shown in FIGS. 3 and 4, reference may be made to the above explanations regarding the first embodiment shown in FIGS. 1 and 2 owing of the features in common between the first and second embodiment.

FIG. 5a) shows a view of an upper side 51 of a third embodiment of a plasma treatment device 1 according to the invention. Further details of the structure of this third embodiment may be derived from the longitudinal section illustrated in FIG. 5b) and the cross sections shown in FIGS. 5c) and 5d).

The third embodiment shown here is similar to the second embodiment of the plasma treatment device 1 explained above. In contrast thereto, the line arrangement 11 of the third embodiment shown here, as may be seen from FIG. 5b), however comprises a plurality of high-voltage supply lines 10, specifically a first high-voltage supply line 10a and a second high-voltage supply line 10b, to which different high-voltage signals are able to be applied. The electrode arrangement 5 in this third embodiment furthermore comprises a plurality of electrodes 3, which form a plurality of electrode groups that are electrically insulated from one another. In the exemplary embodiment shown here, these are the two electrode groups 39a, 39b that are electrically insulated from one another.

In the same way as the second embodiment shown in FIGS. 3 and 4, the electrodes 3 are in this case arranged, as may be seen in FIG. 5d), such that one electrode 3 is arranged centrally on the base surface 21 of the bristle carrier 19 and the other electrodes 3 are arranged in three concentric circles, specifically an inner circle, a central circle and an outer circle, around a center point of the circular cross section of the bristle carrier 19. It may be seen in this case from FIG. 5b) in connection with FIG. 5d) that, in this third embodiment, the centrally arranged electrode 3 and the electrodes 3 arranged on the inner circle form a first electrode group 39a and that the electrodes 3 arranged on the central circle and the outer circle form a second electrode group 39b. The two electrode groups 39a, 39b are in this case electrically insulated from one another.

It may furthermore be seen from FIG. 5b) that the different electrode groups 39a, 39b are connected to different high-voltage supply lines 10a, 10b. In the exemplary embodiment shown here, the first electrode group 39a is connected to the first high-voltage supply line 10a and the second electrode group 39b is connected to the second high-voltage supply line 10b. The different electrode groups 39a, 39b are thereby able to be supplied with different high-voltage signals.

The cross section illustrated in FIG. 5c) furthermore shows that the electrode arrangement 5, in this third embodiment of the plasma treatment device 1 according to the invention, has a plurality of distributor plates 41 that are electrically insulated from one another, specifically two distributor plates 41a, 41b. The different distributor plates 41a, 41b are in this case arranged in the same plane. The first distributor plate 41a has a circular cross section and the second distributor plate 41b has a circular cross section, wherein the first distributor plate 41a is cross-sectionally surrounded by the second distributor plate 41b. The first distributor plate 41a and the second distributor plate 41b are electrically insulated from one another by an insulating layer 42.

It may be seen in the longitudinal section illustrated in FIG. 5b) that the electrodes 3 of the different electrode groups 39a, 39b are connected to different distributor plates 41a, 41b and the different distributor plates 41a, 41b are connected to different high-voltage supply lines 10a, 10b. In this exemplary embodiment, the electrodes 3 of the first electrode group 39a are connected to the first distributor plate 41a and the electrodes 3 of the second electrode group 39b are connected to the second distributor plate 41b. The first distributor plate 41a is connected to the first high-voltage supply line 10a and the second distributor plate 41b connected to the second high-voltage supply line 10b. The distributor plates 41a, 41b are thereby configured to distribute the high-voltage signal to the electrodes 3 connected to the respective distributor plate 41a, 41b.

FIG. 6a) shows a view of an upper side 51 of the third embodiment of the plasma treatment device 1 with the brush head 15 removed. Details of the structure may be seen from the longitudinal section illustrated in FIG. 6b) and the cross sections illustrated in FIGS. 6c) and 6d). It may be seen in this case in FIGS. 6a) and 6b) that, in the third embodiment shown here as well, the plasma treatment device 1 has a handle part 43 and a removable part 45 of the brush head 15, which is able to be connected to the handle part 43 in a detachable and exchangeable manner by way of a mechanical connection arrangement. The removable part 45 of the brush head 15 in this case comprises, in the same way as the second embodiment explained above, the entire electrode arrangement 5 and has a connector that, in the mounted state, makes contact with a contact arrangement of the handle part 43.

For the rest, reference may be made to the explanations with regard to the embodiments explained above owing to the features in common between the third embodiment shown in FIGS. 5 and 6 and the first and the second embodiment.

FIGS. 7a), 7b) and 7c) respectively show a view of an upper side 51, a longitudinal section and a view of a contact side 57 of a fourth embodiment of the plasma treatment device 1 according to the invention with the brush head 15 removed. The fourth embodiment that is illustrated is similar to the first embodiment shown in FIGS. 1 and 2.

FIGS. 8a), 8b) and 8c) respectively show a view of an upper side 51, a longitudinal section and a cross section of a fifth embodiment of a plasma treatment device 1 according to the invention with the brush head 15 removed. The fifth embodiment that is illustrated is similar to the second embodiment shown in FIGS. 3 and 4.

FIGS. 9a), 9b), 9c) and 9d) respectively show a view of an upper side 51, a longitudinal section and two cross sections of a sixth embodiment of a plasma treatment device 1 according to the invention. The sixth embodiment that is illustrated is similar to the third embodiment shown in FIGS. 5 and 6.

As may be seen in FIGS. 7b), 8b) and 9b), in the fourth, fifth and sixth embodiment respectively shown there, the housing 9 also in each case contains an electronic controller 63, an intermediate stage 64 and a high-voltage stage 65, which are arranged in the interior 67 of the housing 9.

In contrast to the embodiments illustrated in FIGS. 1 to 6, the plasma treatment device 1 in the fourth, fifth and sixth embodiment illustrated respectively in FIGS. 7, 8 and 9 however does not have any battery 61 that supplies the supply voltage, but rather is configured to be connected to an external voltage source 75. For this purpose, provided in the housing 9 of the plasma treatment device 1 is a respective cable bushing 79, through which a cable 77 that is able to be connected to the external voltage source 75 is passed. In the exemplary embodiments that are shown, the external voltage source 75 in this case supplies a mains voltage in the form of a low voltage as supply voltage, specifically an AC voltage having a nominal value of 230 V and a frequency of 50 Hz. The supply voltage provided by the voltage source 75 is supplied to the electronic controller 63 via the cable 77 connected to the electronic controller 63. An AC high-voltage signal is generated from the mains voltage by way of the electronic controller 63, the intermediate stage 64 and the high-voltage stage 65. The AC voltage signal thereby generated may, in the same way as the first, second and third embodiment explained above, be supplied to the electrode arrangement 5 via the high-voltage supply lines 10a, 10b in order to supply the at least one electrode 3 with the high-voltage signal.

For the rest, with regard to the fourth, fifth and sixth embodiment shown in FIGS. 7, 8 and 9, reference may be made to the above explanations in this respect in relation to the respective correspondences with the first, second and third embodiments.

FIG. 10a) shows a view of an upper side 51 of a seventh embodiment of a plasma treatment device 1 according to the invention with the brush head 15 removed. Further details of the structure of this seventh embodiment may be derived from the longitudinal section illustrated in FIG. 10b) and the cross sections illustrated in FIGS. 10c) and 10d).

The seventh embodiment that is shown here is similar to the third embodiment of the plasma treatment device 1 according to the invention shown in FIGS. 5 and 6. In the same way as the third embodiment explained above, the line arrangement 11, as may be seen from FIG. 10b), has two high-voltage supply lines, specifically a first high-voltage supply line 10a and a second high-voltage supply line 10b, to which different high-voltage signals are able to be applied. Furthermore, in this embodiment too, the electrode arrangement 5 comprises a plurality of electrodes 3 that form two electrode groups that are electrically insulated from one another, specifically a first electrode group 39a and a second electrode group 39b. The different electrode groups 39a, 39b are in this case connected to different high-voltage supply lines 10a, 10b such that the first electrode group 39a is connected to the first high-voltage supply line 10a and the second electrode group 39b is connected to the second high-voltage supply line 10b. The different electrode groups 39a, 39b are thereby able to be supplied with different high-voltage signals.

The seventh embodiment shown in FIG. 10 differs from the third embodiment explained above, as may be seen in FIG. 10b) in connection with FIG. 10d), through the arrangement of the electrodes 3 and the electrode groups 39a, 39b formed by the electrodes 3. In this seventh embodiment, one electrode 3 of the first electrode group 39a is thus arranged centrally on the base surface 21 of the bristle carrier 19, and the other electrodes 3 of the two electrode groups 39a, 39b are arranged on four concentric circles around the center point of the circular cross section of the bristle carrier 19. These concentric circles are referred to below as first circle, second circle, third circle and fourth circle, wherein the first circle has the smallest diameter, the second circle has the second-smallest diameter, the third circle has the second-largest diameter and the fourth circle has the largest diameter. It may be seen from the longitudinal section illustrated in FIG. 10b) in connection with the cross section illustrated in FIG. 10d) that the electrodes 3 of the first electrode group 39a are arranged on the second and fourth circle, while the electrodes 3 of the second group 39b are arranged on the first and third circle.

The electrodes 3 of the different electrode groups 39a, 39b may accordingly be arranged at different distances from a center point of the base surface 21 of the bristle carrier 19. The electrodes 3 of the different electrode groups 39a, 39b may in particular be arranged on different concentric circles having different diameters around a center point of the base surface 21 of the bristle carrier 19.

The different electrode groups 39a, 39b may accordingly also be arranged alternately on the base surface 21 of the bristle carrier 19. The different electrode groups 39a, 39b may in particular be arranged alternately in the radial direction on the base surface 21 of the bristle carrier 19.

It may furthermore be seen in FIG. 10b that the high-voltage supply lines 10a, 10b branch into a plurality of supply line sections on their side facing the electrode arrangement 5 in order to be able to supply the electrodes 3 of the different electrode groups 39a, 39b with the respective high-voltage signal. In this exemplary embodiment, the first high-voltage supply line 10a in this case branches into three supply line sections, and the second high-voltage line 10b branches into two supply line sections.

The electrode arrangement 5 in this seventh embodiment, as may be seen in FIG. 10c), furthermore has four distributor plates 41a, 41b, 41c and 41d that are electrically insulated from one another, have a circular cross section, have different diameters, are arranged concentrically and are configured to distribute the high-voltage signal to the electrodes 3 connected to the respective distributor plate 41a, 41b, 41c, 41d. The first distributor plate 41a in this case has the smallest diameter, the second distributor plate 41b has the second-smallest diameter, the third distributor plate 41c has the second-largest diameter and the fourth distributor plate 41d has the largest diameter.

In order to distribute the high-voltage signal to the electrodes 3, the first distributor plate 41a and the third distributor plate 41c are connected to the electrodes 3 of the second electrode group 39b, while the second distributor plate 41b and the fourth distributor plate 41d are connected to the electrodes 3 of the first electrode group 39a. Furthermore, for this purpose, the distributor plates 41a, 41b, 41c and 41d are connected, on their sides facing the high-voltage supply lines 10a, 10b, to the supply line sections of the high-voltage supply lines 10a, 10b such that the second distributor plate 41b and the fourth distributor plate 41d are each connected to a supply line section of the first high-voltage supply line 10a, while the first distributor plate 41a and the third distributor plate 41c are each connected to a supply line section of the second high-voltage supply line 10b. The electrode 3 arranged centrally on the base surface 21 of the bristle carrier 19 is connected to a third supply line section of the first high-voltage supply line 10a. It is thereby possible to supply the electrode groups 39a, 39b arranged alternately on the bristle carrier 19 with different high-voltage signals, as will be explained by way of example in more detail below with reference to FIGS. 11a) and 11b).

FIG. 11a) shows a perspective view of components of the seventh embodiment, already explained above, of the plasma treatment device 1 according to the invention having the two electrode groups 39a, 39b that are supplied with AC high-voltage signals 13a, 13b in phase opposition. FIG. 11b) shows a longitudinal section of the components illustrated in FIG. 11a).

The first high-voltage supply line 10a, which is branched into three line sections, and the second high-voltage supply line 10b, which is branched into two line sections, are shown. As has already been explained in connection with FIG. 10b), the first high-voltage supply line 10a is in this case connected to the electrode 3 arranged centrally on the base surface 21 of the bristle carrier 19, which is part of the first electrode group 39a, as well as to the second distributor plate 41b and the fourth distributor plate 41d. The second distributor plate 41b and the fourth distributor plate 41d in this case distribute the high-voltage signal 13a applied to the high-voltage supply line 10a to the electrodes 3 of the first electrode group 39a. All of the electrodes 3 of the first electrode group 39a may thereby be supplied with the high-voltage signal 13a applied to the high-voltage supply line 10a.

The second high-voltage supply line 10b is connected to the first distributor plate 41a and the third distributor plate 41c. The first distributor plate 41a and the third distributor plate 41c in this case distribute the high-voltage signal 13b applied to the second high-voltage supply line 10b to the electrodes 3 of the second electrode group 39b. All of the electrodes 3 of the second electrode group 39b may thereby be supplied with the high-voltage signal 13b applied to the high-voltage supply line 10b.

FIGS. 11a) and 11b) also schematically illustrate that the high-voltage signals 13a, 13b are in the form of AC high-voltage signals. The AC high-voltage signals 13a, 13b are in this case in phase opposition to one another and have substantially the same peak value. The first AC high-voltage signal 13b is accordingly phase-shifted by approximately 180 degrees with respect to the first AC high-voltage signal 13a. This achieves a situation whereby the different electrode groups 39a, 39b are supplied with AC high-voltage signals 13a, 13b in phase opposition such that the first electrode group 39a is supplied with the first AC high-voltage signal 13a and the second electrode group 39b is supplied with the second AC high-voltage signal 13b that is in phase opposition with respect to the first AC high-voltage signal 13a.

FIG. 12a) shows a perspective view of components of an eighth embodiment of a plasma treatment device 1 according to the invention having two electrode groups 39a, 39b that are supplied with AC high-voltage signals 13a, 13b in phase opposition. FIG. 12b) shows a longitudinal section of the components illustrated in FIG. 12a).

It may be seen that the electrode arrangement 5, in this eighth embodiment of the plasma treatment device 1, has two distributor plates 41a, 41b to which AC high-voltage signals 13a, 13b in phase opposition are supplied. The first distributor plate 41a is in this case connected to the electrodes 3 of the first electrode group 39a and the second distributor plate 41b is connected to the electrodes of the second electrode group 39b. The different electrode groups 39a, 39b are thereby able to be supplied with different high-voltage signals, specifically with the AC high-voltage signals 13a, 13b in phase opposition.

In contrast to the embodiments explained above, the distributor plates 41a, 41b in this eighth embodiment are however not arranged in the same plane, but in different planes. In this exemplary embodiment, the distributor plates 41a, 41b are arranged one behind the other as seen from the contact surface 27. In this case, the second distributor plate 41b is an inner distributor plate and the first distributor plate 41a is an outer distributor plate. The second distributor plate 41b is arranged between the first distributor plate 41a and the bristle field 17. In order to allow a connection between the electrodes extending into the bristle field 17 and the outer first distributor plate 41a, which is arranged behind the inner second distributor plate 41b as seen from the contact surface 27, the inner second distributor plate has a plurality of through-holes 81 through which the electrodes connected to the outer first electrode 41a extend.

### LIST OF REFERENCE SIGNS

1 plasma treatment device
3 electrode
5 electrode arrangement
7 dielectric
9 housing
10, 10a, 10b high-voltage supply line
11 line arrangement
13a, 13b high-voltage signal
15 brush head
17 bristle field
19 bristle carrier
21 base surface
23 bristles
24 bristle bundle
25 intermediate space
27 contact surface
29 through-opening
31 electrode finger
33 dielectric layer
35 flat end face
37 flat end wall
39a, 39b electrode group
41, 41a, 41b, 41c, 41d distributor plate
42 insulating layer
43 handle part
45 removable part
47 spacers
49 anchoring area
51 upper side
53 lower side
55 rear side
57 contact side
59 control line
61 battery
63 electronic controller
64 intermediate stage
65 high-voltage stage
67 interior
71 contact arrangement
73 connector
75 external voltage source
77 cable
79 cable bushing
81 through-hole

1. A plasma treatment device designed to treat a surface with a dielectric barrier plasma, comprising:
an electrode arrangement that has at least one electrode;
a dielectric that completely covers the at least one electrode in a direction of the surface to be treated;
a housing that contains a line arrangement comprising at least one high-voltage supply line, wherein the at least one electrode is connected to the line arrangement and is able to be supplied by the at least one high-voltage supply line, with a high-voltage signal that is able to be applied to the at least one high-voltage supply line;
a brush head that has a bristle field and a bristle carrier having a base surface, wherein the bristle field has a multiplicity of flexible bristles and intermediate spaces between the bristles, wherein the bristles protrude from the base surface of the bristle carrier in a direction of a contact surface that is defined by ends of longest bristles of the bristle field that face away from the base surface,
wherein the bristle field has a first length defined by a distance between the base surface and the contact surface, and
wherein the at least one electrode of the electrode arrangement extends from the base surface with a second length that is less than the first length or equal to the first length and at least 30% of the first length, into the bristle field in the direction of the contact surface.

2. The plasma treatment device as claimed in claim 1, wherein the at least one electrode of the electrode arrangement extends into the intermediate spaces in the bristle field.

3. The plasma treatment device as claimed in claim 1 wherein the at least one electrode of the electrode arrangement extends into at least one bristle.

4. The plasma treatment device as claimed in claim 1 wherein the at least one electrode of the electrode arrangement consists of a castable plastic provided with conductive additives.

5. The plasma treatment device as claimed in claim 1 wherein at least some of the bristles are formed partially or completely from the dielectric.

6. The plasma treatment device as claimed in claim 1 wherein the electrode arrangement is partially embedded in the bristle carrier, wherein the at least one electrode of the electrode arrangement protrudes from the bristle carrier in the direction of the contact surface.

7. The plasma treatment device as claimed in claim 1 wherein the bristle carrier has a through-opening through which the at least one electrode of the electrode arrangement extends.

8. The plasma treatment device as claimed in claim 1 wherein the at least one electrode of the electrode arrangement is flexible.

9. The plasma treatment device as claimed in claim 1 wherein the at least one electrode in the electrode arrangement comprises a plurality of finger-shaped electrodes that are surrounded by a dielectric layer and protrude from the base surface of the bristle carrier into the bristle field.

10. The plasma treatment device as claimed in claim 1 wherein the at least one electrode of the electrode arrangement which extends into the bristle field has a flat end face facing the contact surface, wherein the flat end face is covered by a flat end wall of the dielectric in the direction of the contact surface.

11. The plasma treatment device as claimed in claim 1 wherein some or all of the bristles of the bristle field on a side of the bristle field facing the contact surface protrude beyond the at least one electrode of the electrode arrangement which extends into the bristle field and the dielectric covering the at least one electrode.

12. The plasma treatment device as claimed in claim 1 wherein the second length is at least 95% of the first length.

13. The plasma treatment device as claimed in claim 1 wherein the bristle carrier is mounted so as to be movable, and further comprises a drive unit configured to drive a movement of the bristle carrier.

14. The plasma treatment device as claimed in claim 1 wherein the line arrangement comprises a plurality of high-voltage supply lines to which different high-voltage signals are able to be applied, and wherein the at least one electrode of the electrode arrangement comprises a plurality of electrodes that form a plurality of electrode groups that are electrically insulated from one another, wherein different electrode groups are connected to different high-voltage supply lines and are able to be supplied with different high-voltage signals.

15. The plasma treatment device as claimed in claim 14, wherein the electrode arrangement has a plurality of electrically conductive distributor plates that are electrically insulated from one another, wherein each distributor plate is connected to a plurality of electrodes and to one of the high-voltage supply lines and is configured to distribute the high-voltage signal to the plurality of electrodes connected to the distributor plate, and wherein the plurality of electrodes of the different electrode groups are connected to different distributor plates and the different distributor plates are connected to different high-voltage supply lines.

16. The plasma treatment device as claimed in claim 1 further comprising a handle part and at least one removable part of the brush head connected to the handle part in a detachable and exchangeable manner by way of a mechanical connection arrangement, wherein the at least one removable part comprises the bristle carrier and the bristle field.

17. A brush head of a plasma treatment device as claimed in claim 1.

18. A removable part of a brush head of a plasma treatment device as claimed in claim 16.

19. The plasma treatment device of claim 5 wherein the at least some of the bristles are bristles into which the at least one electrode of the electrode arrangement extends.

20. The plasma treatment device of claim 13 wherein the drive unit drives the bristle carrier to move in one or more of a rotational movement and a rotational oscillating movement.
